(19) United States
(12) Patent Application Publication
(10) Pub. No.: US 2026/0249020 A1
(43) Pub. Date: Aug. 27, 2026

(54) SYRINGE CONNECTOR FOR A MEDICAMENT DELIVERY DEVICE

(71) Applicant: Genzyme Corporation, Cambridge, MA (US)

(72) Inventors: Alexander Hee-Hanson, Melbourn (GB); Thomas Lever, Melbourn (GB); Michael Parrott, Melbourn (GB); Robert Wilson, Melbourn (GB); Haiming Wu, Cambridge, MA (US)

(21) Appl. No.: 19/063,630

(22) Filed: Feb. 26, 2025

(51) Int. Cl. A61M 5/34

(52) U.S. Cl. CPC A61M 5/344

(57) ABSTRACT

The application relates to a syringe connector for a medicament delivery device. The syringe connector comprises a main body having a longitudinal axis. The main body comprises a first wall extending transversely to the longitudinal axis, an opposing second wall extending transversely, and a side wall connecting the first and second walls. The walls define a cavity configured to receive a proximal end of a syringe. The cavity comprises a syringe flange receiving portion located between the first and second walls and delimited by the side wall, and a syringe body receiving portion formed by an aperture extending through the second wall. The cavity extends to an opening in the side wall and the edge of the second wall such that a proximal end of a syringe can be inserted transversely into the cavity. The syringe connector further comprises a syringe retention element and an impact force reduction element.

### TECHNICAL FIELD

The present application relates to a syringe connector for a medicament delivery device, a medicament delivery device comprising a syringe connector, and a method of forming a syringe connector.

### BACKGROUND

A syringe connector is a component of a medicament delivery device that is designed to be located around a proximal end of a syringe. The syringe connector is utilised mainly during the dispensing step such that a spring or spring guide contacts the syringe connector.

Known syringe connectors often have geometries and features that can only be formed by multiple moulding steps, which require complex tooling apparatus to achieve. Complex tooling apparatus can be expensive and prone to malfunction.

### SUMMARY

According to a first aspect, there is provided a syringe connector for a medicament delivery device, the syringe connector comprising a main body having a longitudinal axis, the main body comprising a first wall extending transversely to the longitudinal axis, an opposing second wall extending transversely, and a side wall connecting the first and second walls, the walls defining a cavity configured to receive a proximal end of a syringe, wherein the cavity comprises a syringe flange receiving portion located between the first and second walls and delimited by the side wall, and a syringe body receiving portion formed by an aperture extending through the second wall, wherein the cavity extends to an opening in the side wall and the edge of the second wall such that a proximal end of a syringe can be inserted transversely into the cavity, a syringe retention element configured to restrict radial movement of a syringe when a syringe is located in the syringe connector, and an impact force reduction element configured to reduce force transmitted to a syringe when a dispensing mechanism is triggered, during use.

The syringe connector may protect the syringe from excessive impact forces during transportation and use of a medicament delivery device. The syringe retention element may prevent the syringe from rattling inside the device and the impact force reduction element may reduce the dispensing mechanism forces imparted to the syringe. Each of the syringe retention element and the impact force reduction element may be individually tailored to the requirements of the device and medicament to be delivered.

In some embodiments, the syringe retention element may comprise a pair of arms forming a syringe clip, the arms being located on opposing sides of the cavity and extending from the main body into the cavity to define a central syringe retention area within the cavity.

The pair of arms may hold and retain a syringe in the syringe retention area in order to maintain a syringe in its correct alignment. The pair of arms may reduce the amount that a syringe rattles within the cavity of the syringe connector and reduce the chances of damage being caused to a syringe.

In some embodiments, the pair of arms may be configured such that a minimum distance between the arms is smaller than a diameter of the syringe retention area.

In some embodiments, each arm of the pair of arms may be resiliently flexible in a radially outward direction such that the arms deform when a syringe is moved through the opening into the syringe retention area.

A syringe cannot freely move in and out of the syringe retention area. Thus, a syringe can be retained in the syringe retention area unless a predetermined force is applied to move the arms apart. This reduces accidental decoupling of a syringe from the syringe connector.

In some embodiments, the pair of arms may extend from an inner surface of the second wall into the cavity and are configured to contact a syringe body to retain a syringe when a syringe is received in the syringe retention area.

The pair of arms may retain the syringe and reduce radial movement by contact with the syringe body. The pair of arms may also reduce axial movement by contact with the syringe body directly under a flange of the syringe. The pair of arms may abut the underside of the flange of the syringe to reduce axial movement of the syringe.

In some embodiments, the pair of arms may extend from the side wall into the syringe flange receiving portion and are configured to contact a syringe flange and retain a syringe when a syringe is received in the syringe retention area.

The pair of arms may be located closer to the first wall of the syringe connector and retain the syringe. The location of the arms closer to the first wall provides greater space for the accommodation of the impact force reduction element.

In some embodiments, each arm of the pair of arms may comprise a radially innermost section configured to abut a side of a syringe proximal to the opening a restrict radial movement of a syringe when a syringe is received in the syringe retention area.

The inner surface of the arm being contoured to match the syringe body reduces the amount of rattle of the syringe in the syringe connector. In addition, the contact area between the syringe and the syringe connector is increased, which minimises contact pressure on the syringe and reduces the likelihood of the syringe becoming damaged.

In some embodiments, each arm of the pair of arms may comprise a second section located radially outwards of the innermost section, each second section comprising a guide surface configured to guide a syringe towards the syringe retention area during syringe insertion.

The guide surfaces may result is a larger positional tolerance for assembly of the syringe in the syringe connector. That is, the guide surfaces remove the low tolerance on accuracy required for assembly.

In some embodiments, the innermost section and the second section of each arm may join at a ridge, the gap between the opposing ridges being the minimum distance between the arms, wherein the guide surfaces diverge with distance in the radial direction.

The ridge may help to retain the syringe in the syringe retention area to prevent, or at least reduce the likelihood, of accidentally decoupling of the syringe and the syringe connector.

In some embodiments, the innermost section and the second section may both extend from the inner surface of the second wall to form an enclosed aperture between each arm and the second wall, each arm being configured to deform into its respective aperture when a syringe is inserted into the syringe retention area.

The enclosed aperture may reduce the force required to deflect the arms during coupling of the syringe to the syringe connector. By reducing this assembly force, the chance of causing damage to the syringe or permanent deformation of the syringe connector can be reduced.

In some embodiments, the innermost section may extend from the inner surface of the second wall and the second section comprises a free end, an open ended slot located between each arm and the second wall, wherein the open end of the slot is distal to and facing away from the syringe retention area, each arm being configured to deform into its respective slot when a syringe is inserted into the syringe retention area.

The open-ended slot may provide an even greater reduction in deflection force due to a large moment arm. The force required to insert the syringe may increase with decreasing distance to the syringe retention area. Thus, the ease of assembly is increased and the chance of damaging the syringe or permanently deforming the arm is reduced. The tooling for such an embodiment is also easier to produce and can be moved in the axial direction or the radial direction during the manufacturing process due to the open-ended slot.

In some embodiments, the second section may extend from the inner surface of the second wall and the innermost section comprises a free end, an open ended slot located between each arm and the second wall, wherein the open end of the slot is proximate to and facing toward the syringe retention area, wherein each arm is configured to deform into its respective slot when a syringe is inserted into the syringe retention area.

The open-ended slot may provide a greater reduction in deflection force. The force required to insert the syringe may decrease with decreasing distance to the syringe retention area. Thus, the ease of assembly is increased and the chance of damaging the syringe or permanently deforming the arm is reduced.

In some embodiments, the impact force reduction element may be located outside of the main body longitudinally adjacent to and extending from the second wall, the impact force reduction element being configured to resiliently deform to absorb impact energy, during use.

Thus, the impact force reduction element may be located between the main body of the syringe connector and the collar into which the syringe connector is driven by the dispensing mechanism. As a result, the impact force reduction element can deform on contact with collar to reduce the amount of force transferred from impact of the syringe connector with the collar to a syringe.

In some embodiments, the impact force reduction element may comprise at least one transversely extending cantilevered arm longitudinally spaced from the second wall by a transversely extending slot, the at least one cantilevered arm comprising a contact projection located distally to the longitudinal axis and extending in the longitudinal direction away from the main body.

The cantilevered arm may flex to absorb impact energy. The contact projection may increase the amount of deflection of the cantilevered arm before the collar contact the arm. Thus, more energy can be absorbed before the collar contact the main body of the syringe connector.

In some embodiments, the impact force reduction element may comprise a pair of deformable arms extending from opposing sides of the syringe body receiving portion of the cavity, wherein the arms extend parallel to each other, and each arm of the pair of arms comprises an inclined portion whose distance from the longitudinal direction of the main axis increases with distance from the second wall.

The deformable arms may flex to absorb impact energy. The inclined portion of the arms result in a larger amount of energy being absorbed before the collar contact the main body of the syringe connector.

In some embodiments, the impact force reduction element may be formed by the second wall, wherein the second wall is resiliently deformable in the longitudinal direction to absorb impact energy, due use.

In some embodiments, the components of the syringe connector may be an integrally formed monolithic structure.

The components of the syringe connector 400 being formed integrally lowers the unit cost. In addition, the manufacture process is quicker due to only requiring one injection moulding step and removes the need for sub-assembly apparatus.

In some embodiments, the syringe connector may be formed from a thermoplastic material or an elastomeric material.

The thermoplastic material may comprise the material strength required to maintain the structure of the syringe connector whilst providing the flexibility required to allow the syringe retention element to resiliently deform and the impact force reduction element to deform to absorb impact energy. The elastomeric may provide enhanced impact energy absorbing properties. Both materials may be suitable for injection moulding manufacturing processes.

In some embodiments, the syringe connector may further comprise an insert located on the internal transverse surface of the second wall.

In some embodiments, the insert may comprise the syringe retention element in the form of a pair of ridges extending into the cavity to define a syringe retention area.

In some embodiments, the insert may comprise the impact force reduction element in the form of the insert being formed by an elastomeric material that is configured to deform to absorb energy during use.

In some embodiments, the insert may comprise an O-ring configured to surround a proximal end of a syringe body and abut a flange of a syringe.

In some embodiments, the insert may be formed from a moulded elastomer.

In a second aspect, there is provided a medicament delivery device comprising a syringe connector according to any one of claims 1 to claim 18.

In a third aspect, there is provided a method of forming a syringe connector according to any one of claim 1 to claim 18, the method comprising the steps of positioning a plurality of mould parts to form a syringe connector shaped cavity, providing a material injection port in at least one of the plurality of mould parts, performing a single injection of material into the syringe connector shaped cavity to fill the syringe connector shaped cavity, and cooling the material to form the syringe connector.

The manufacture of the syringe connector takes place in a single injection step. Thus, the time and cost of manufacture of the syringe connector is reduced.

In some embodiments, the method may further comprise providing an insert and locating the insert of an inner transversely extending surface of the second wall.

### BRIEF DESCRIPTION OF THE DRAWINGS

Embodiments will now be described, by way of example only, with reference to the accompanying drawings, in which:

FIG. 1A shows a schematic view of a medicament delivery device with a cap attached;

FIG. 1B shows a schematic view of the medicament delivery device of FIG. 1A with the cap removed;

FIG. 2A shows a schematic view of a medicament delivery device prior to use (i.e. in a pre-use configuration);

FIG. 2B shows a schematic view of the device of FIG. 2A with the cap removed;

FIG. 2C shows a schematic view of the device of FIG. 2A showing the device placed at an injection site;

FIG. 2D shows a schematic view of the device of FIG. 2A with the button having been pressed to release the dispensing mechanism;

FIG. 2E shows a schematic view of the device of FIG. 2A with the button having been pressed to release the dispensing mechanism;

FIG. 2F shows a schematic view of the device of FIG. 2A showing the needle having retracted within the device after a dose has been delivered;

FIG. 2G shows a schematic view of the device of FIG. 2A showing the device removed from the injection site after the needle has retracted within the device after delivery of the medicament;

FIG. 3 shows a schematic perspective view of a known syringe connector;

FIG. 4 shows a schematic cross-sectional side view of a moulding apparatus for forming a syringe connector;

FIG. 5A shows a schematic view perspective top view of a syringe connector with a syringe located therein;

FIG. 5B shows a schematic perspective top view of the syringe connector of FIG. 5A;

FIG. 5C shows a schematic perspective bottom view of the syringe connector of FIG. 5A;

FIG. 5D shows a schematic cross-sectional side view of the syringe connector of FIG. 5A;

FIG. 6A shows a schematic view perspective top view of a syringe connector with a

FIG. 6B shows a schematic perspective top view of the syringe connector of FIG. 6A;

FIG. 6C shows a schematic perspective bottom view of the syringe connector of FIG. 6A;

FIG. 6D shows a schematic cross-sectional side view of the syringe connector of FIG. 6A;

FIG. 7A shows a schematic view perspective top view of a syringe connector with a syringe located therein;

FIG. 7B shows a schematic perspective top view of the syringe connector of FIG. 7A;

FIG. 7C shows a schematic perspective bottom view of the syringe connector of FIG. 7A;

FIG. 7D shows a schematic cross-sectional side view of the syringe connector of FIG. 7A;

FIG. 8A shows a schematic view perspective top view of a syringe connector with a syringe located therein;

FIG. 8B shows a schematic perspective top view of the syringe connector of FIG. 8A;

FIG. 8C shows a schematic perspective bottom view of the syringe connector of FIG. 8A;

FIG. 8D shows a schematic cross-sectional side view of the syringe connector of FIG. 8A;

FIG. 9A shows a schematic view perspective top view of a syringe connector with a syringe located therein;

FIG. 9B shows a schematic perspective top view of the syringe connector of FIG. 9A;

FIG. 9C shows a schematic perspective bottom view of the syringe connector of FIG. 9A;

FIG. 9D shows a schematic cross-sectional side view of the syringe connector of FIG. 9A;

FIG. 10A shows a schematic view perspective top view of a syringe connector with a syringe located therein;

FIG. 10B shows a schematic perspective top view of the syringe connector of FIG. 10A;

FIG. 10C shows a schematic perspective bottom view of the syringe connector of FIG. 10A;

FIG. 10D shows a schematic cross-sectional side view of the syringe connector of FIG. 10A;

FIG. 11A shows a schematic view perspective top view of a syringe connector with a syringe located therein;

FIG. 11B shows a schematic perspective top view of the syringe connector of FIG. 11A;

FIG. 11C shows a schematic perspective bottom view of the syringe connector of FIG. 11A;

FIG. 11D shows a schematic cross-sectional side view of the syringe connector of FIG. 11A;

FIG. 12 shows a schematic perspective top view of a syringe connector; and

FIG. 13 shows a schematic perspective top view of a syringe connector.

### DETAILED DESCRIPTION

A drug delivery device, as described herein, may be configured to inject a medicament into a patient. For example, delivery could be sub-cutaneous, intra-muscular, or intravenous. Such a device could be operated by a patient or care-giver, such as a nurse or physician, and can include various types of safety syringe, pen-injector, or auto-injector. The device can include a cartridge-based system that requires piercing a sealed ampule before use. Volumes of medicament delivered with these various devices can range from about 0.5 ml to about 2 ml. Yet another device can include a large volume device (“LVD”) or patch pump, configured to adhere to a patient's skin for a period of time (e.g., about 5, 15, 30, 60, or 120 minutes) to deliver a “large” volume of medicament (typically about 2 ml to about 10 ml).

In combination with a specific medicament, the presently described devices may also be customized in order to operate within required specifications. For example, the device may be customized to inject a medicament within a certain time period (e.g., about 3 to about 20 seconds for auto-injectors, and about 10 minutes to about 60 minutes for an LVD). Other specifications can include a low or minimal level of discomfort, or to certain conditions related to human factors, shelf-life, expiry, biocompatibility, environmental considerations, etc. Such variations can arise due to various factors, such as, for example, a drug ranging in viscosity from about 3 cP to about 50 cP. Consequently, a drug delivery device will often include a hollow needle ranging from about 25 to about 31 Gauge in size. Common sizes are 27 and 29 Gauge.

The delivery devices described herein can also include one or more automated functions. For example, one or more of needle insertion, medicament injection, and needle retraction can be automated. Energy for one or more automation steps can be provided by one or more energy sources. Energy sources can include, for example, mechanical, pneumatic, chemical, or electrical energy. For example, mechanical energy sources can include springs, levers, elastomers, or other mechanical mechanisms to store or release energy. One or more energy sources can be combined into a single device. Devices can further include gears, valves, or other mechanisms to convert energy into movement of one or more components of a device.

The one or more automated functions of an auto-injector may each be activated via an activation mechanism. Such an activation mechanism can include one or more of a button, a lever, a needle sleeve, or other activation component. Activation of an automated function may be a one-step or multi-step process. That is, a user may need to activate one or more activation components in order to cause the automated function. For example, in a one-step process, a user may depress a needle sleeve against their body in order to cause injection of a medicament. Other devices may require a multi-step activation of an automated function. For example, a user may be required to depress a button and retract a needle shield in order to cause injection.

In addition, activation of one automated function may activate one or more subsequent automated functions, thereby forming an activation sequence. For example, activation of a first automated function may activate at least two of needle insertion, medicament injection, and needle retraction. Some devices may also require a specific sequence of steps to cause the one or more automated functions to occur. Other devices may operate with a sequence of independent steps.

Some delivery devices can include one or more functions of a safety syringe, pen-injector, or auto-injector. For example, a delivery device could include a mechanical energy source configured to automatically inject a medicament (as typically found in an auto-injector) and a dose setting mechanism (as typically found in a pen-injector).

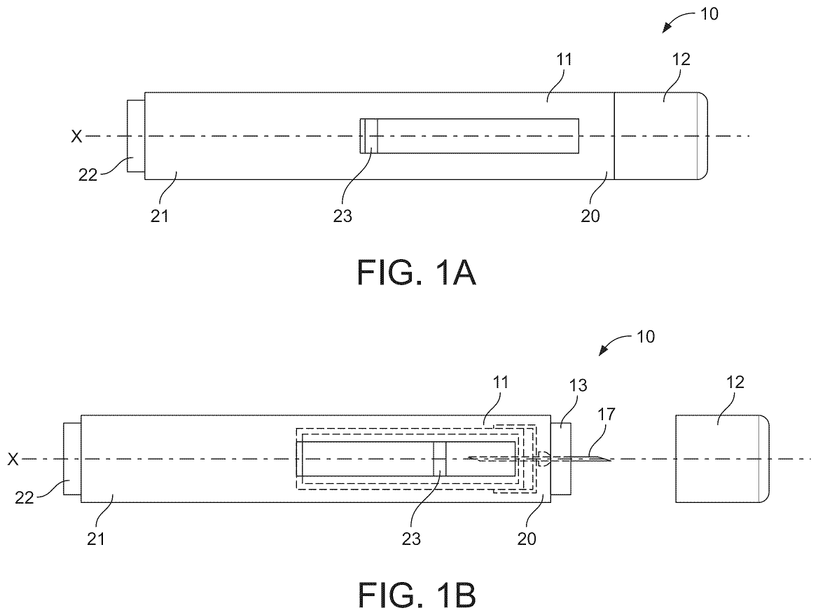

According to some embodiments of the present disclosure, an exemplary drug delivery device 10 is shown in FIGS. 1A & 1B. The device 10, as described above, is configured to inject a medicament into a patient's body. The device 10 includes a housing 11 which typically contains a reservoir containing the medicament to be injected (e.g., a syringe) and the components required to facilitate one or more steps of the delivery process. The device 10 can also include a cap assembly 12 that can be detachably mounted to the housing 11. A user typically removes the cap assembly 12 from the housing 11 before the device 10 is operated.

As shown, the housing 11 is substantially cylindrical and has a substantially constant diameter along the longitudinal axis X. The housing 11 has a distal region 20 and a proximal region 21. The term “distal” refers to a location that is relatively closer to a site of injection, and the term “proximal” refers to a location that is relatively further away from the injection site.

The device 10 can also include a needle sleeve 13 coupled to the housing 11 to permit movement of the sleeve 13 relative to the housing 11. For example, the sleeve 13 can move in a longitudinal direction parallel to the longitudinal axis X. Specifically, movement of the sleeve 13 in a proximal direction can permit a needle 17 to extend from distal region 20 of the housing 11.

Insertion of the needle 17 can occur via several mechanisms. For example, the needle 17 may be fixedly located relative to the housing 11 and initially be located within an extended needle sleeve 13. Proximal movement of the sleeve 13 by placing a distal end of the sleeve 13 against a patient's body and moving the housing 11 in a distal direction will uncover the distal end of the needle 17. Such relative movement allows the distal end of the needle 17 to extend into the patient's body. Such insertion is termed “manual” insertion as the needle 17 is manually inserted via the patient's manual movement of the housing 11 relative to the sleeve 13.

Another form of insertion is “automated,” whereby the needle 17 moves relative to the housing 11. Such insertion can be triggered by movement of the sleeve 13 or by another form of activation, such as, for example, a button 22. As shown in FIGS. 1A & 1B, button 22 is located at a proximal end of the housing 11. However, in other embodiments, the button 22 could be located on a side of the housing 11.

Other manual or automated features can include drug injection or needle retraction, or both. Injection is the process by which a bung or piston 23 is moved from a proximal location within a syringe (not shown) to a more distal location within the syringe in order to force a medicament from the syringe through the needle 17. In some embodiments, a drive spring (not shown) is under compression before the device 10 is activated. A proximal end of the drive spring can be fixed within the proximal region 21 of the housing 11, and a distal end of the drive spring can be configured to apply a compressive force to a proximal surface of the piston 23. Following activation, at least part of the energy stored in the drive spring can be applied to the proximal surface of the piston 23. This compressive force can act on the piston 23 to move it in a distal direction. Such distal movement acts to compress the liquid medicament within the syringe, forcing it out of the needle 17.

Following injection, the needle 17 can be retracted within the sleeve 13 or the housing 11. Retraction can occur when the sleeve 13 moves distally as a user removes the device 10 from a patient's body. This can occur as the needle 17 remains fixedly located relative to the housing 11. Once a distal end of the sleeve 13 has moved past a distal end of the needle 17, and the needle 17 is covered, the sleeve 13 can be locked. Such locking can include locking any proximal movement of sleeve 13 relative to the housing 11.

Another form of needle retraction can occur if the needle 17 is moved relative to the housing 11. Such movement can occur if the syringe within the housing 11 is moved in a proximal direction relative to the housing 11. This proximal movement can be achieved by using a retraction spring (not shown), located in the distal region 20. A compressed retraction spring, when activated, can supply sufficient force to the syringe to move it in a proximal direction. Following sufficient retraction, any relative movement between the needle 17 and the housing 11 can be locked with a locking mechanism. In addition, the button 22 or other components of the device 10 can be locked as required.

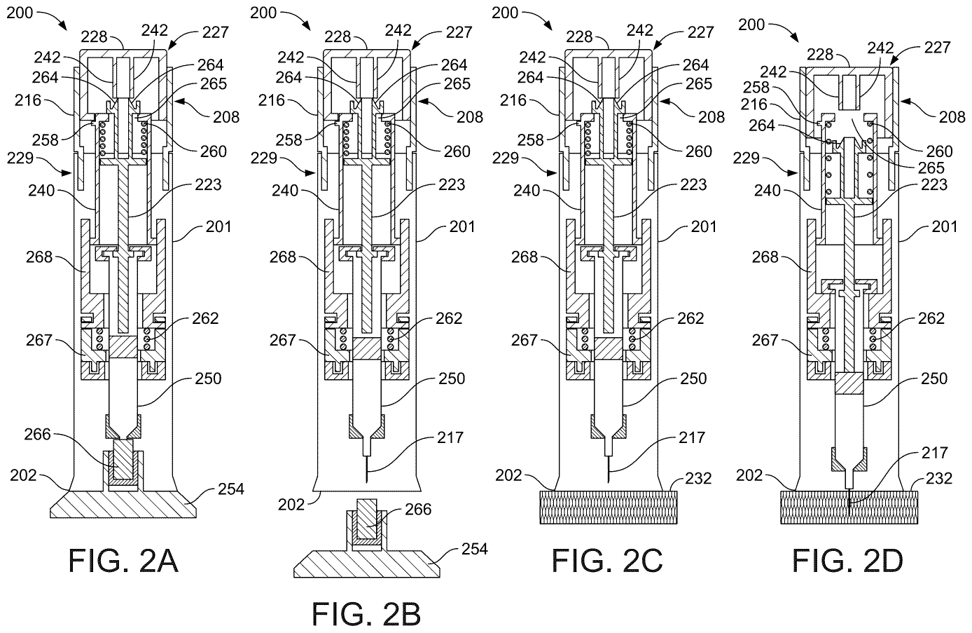

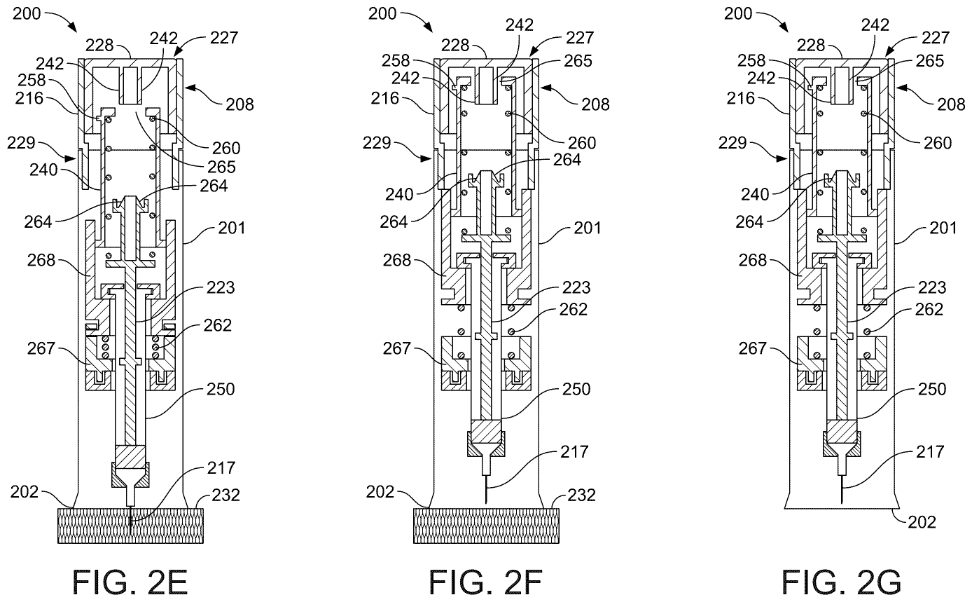

FIGS. 2A to 2G show the sequential steps of operating a medicament delivery device 200. The medicament delivery device is an autoinjector.

The medicament delivery device 200 comprises a body 201, a syringe 250 having a needle 217, and an axially moveable plunger 233 for dispensing medicament from the syringe 250. The medicament delivery device 200 comprises a cap 254 which is removably attached to the body 201 and covers a distal end 202 of the body 201 for preventing access to the needle 217. The medicament delivery device 200 has a needle shield 266 that covers the needle 217 before use. The needle shield 266 is attached to the cap 254.

The medicament delivery device 200 has a dispensing mechanism 229. The medicament delivery device 200 has an actuation member 227 which is configured to release the dispensing mechanism 229. The actuation member 227 is configured to engage the dispensing mechanism 229 to release the dispensing mechanism 229.

The dispensing mechanism 229 is configured to cause the needle 217 to move distally from a needle pre-use position, in which the needle 217 is recessed within the body 201, to an injection position, in which the needle 217 protrudes from the distal end 202 of the body 201 when the dispensing mechanism 229 is released.

The dispensing mechanism 229 is configured to dispense the medicament from the needle 217 when the needle 217 is in the injection position.

As shown in FIGS. 2B and 2C, in order to deliver a dose of medicament to an injection site, the cap 254 is removed (as shown in FIG. 2B) and the medicament delivery device 200 is placed at an injection site 232 (as shown in FIG. 2C).

The actuation member 227 comprises a button 228 and is prevented from being depressed by a stop 258. The stop 258 is provided on the spring guide 240, for example.

The medicament delivery device 200 has a locking member 208 in the form of a lock ring 216 which is rotatable by a user about a longitudinal axis of the medicament delivery device 200. The actuation member 227 is keyed to the lock ring 216 so that the actuation member 227 rotates with the lock ring 216. The lock ring 216 is rotatable from a pre-use position, in which distal movement of the button 228 is prevented, to a use position, in which distal movement of the button 228 is permitted.

When the lock ring 216 is in the pre-use position then the stop 258 engages the button 228 to prevent the button 228 from being depressed.

In order to allow the button 228 to be depressed, the lock ring 216 is rotated about the longitudinal axis of the medicament delivery device 200 from the pre-use position to the use position. The rotation of the lock ring 216 also rotates the actuation member 227 to apposition in which the stop 258 no longer prevents the button 228 from being depressed as shown, for example, in FIG. 2C.

Turning now to FIG. 2D, the user then presses the button 228 to release the dispensing mechanism 229 for dispensing medicament from the medicament delivery device 200. The dispensing mechanism 229 has a plunger 233 and a bias in the form of a compression spring 260. The plunger 223 is biased distally by the spring 260.

The dispensing mechanism 229 is at least partially housed within the spring guide 240. The plunger 223 has a release member, which has proximally-extending clips 264. The spring 260 is retained in the compressed position by virtue of the clips 264, which protrude through a proximal opening 265 in the spring guide 240. The clips 264 engage the spring guide 240 for maintaining the plunger 223 in a proximal position.

The actuation member 227 has a firing member comprising a pair of protrusions 242 which engage with the clips 264 when the button 228 is depressed to flex the clips 264 radially inwardly thereby allowing the clips 264 to move distally through the proximal opening 265 to release the spring 260.

When the dispensing mechanism 229 is released, then the syringe 250 is released for distal axial movement towards the injection site 232 such that the needle 217 moves from the needle pre-use retracted position to an exposed (or “uncovered” or “injection” position for delivering medicament to the injection site 232 under the biasing force of the compression spring 260.

Depressing the button 228 releases the plunger 223, which, biased by the bias 260, moves along the syringe 250 towards the distal end of the medicament delivery device 200 to force medicament within the syringe 250 through the needle 217, thereby delivering a dose of medicament as shown, for example in FIG. 2E.

As shown in FIG. 2F, once the dose of medicament has been delivered, a medicament container bias 262, embodied by a further spring 262, then causes the needle 217 to move axially back to the retracted position, away from the injection site 232 in a proximal direction. The plunger 223 flexes a clip (not shown) on a first collar 267, which allows the first collar 267 to rotate relative to the body 201 and relative to a second collar 268. The first collar 267 rotates from a first position, in which the second collar 268 is axially coupled to the first collar 267, into a second position, in which the second collar 268 is free to move axially relative to the first collar 267. For example, the second collar 268 may comprise a radially protruding coupling element configured to be received in or engage with a corresponding receiving portion of the first collar 267, such that rotating the first collar 267 from the first position into the second position causes the coupling element to be moved out from the receiving portion, to allow the second collar 268 to move axially relative to the first collar 267. Axial movement of the second collar 268 permits the needle 217 to be retracted.

As shown in FIG. 2G, the medicament delivery device 200 is then removed from the injection site 232, for disposal.

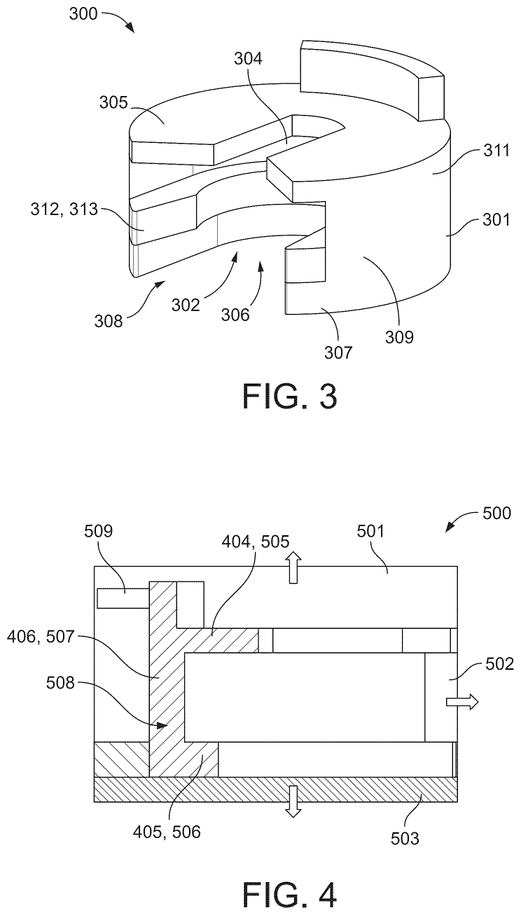

FIG. 3 is a schematic perspective top view of a known syringe connector 300. The syringe connector 300 comprises a main body 301. The main body 301 is generally cylindrical. The main body 301 comprises a syringe receiving recess 302 therein. The syringe receiving recess 302 is configured to receive a proximal end of syringe (not shown).

The syringe receiving recess 302 extends to an opening 304 in a top wall 305 of the main body 301. The opening 304 in the top wall 305 is configured to allow a plunger rod (not shown) to extend therethrough to contact and move a plunger, also known as a piston or bung, during use.

The syringe receiving recess 302 extends to an opening 306 in a bottom wall 307 of the main body 301. The opening 306 in the bottom wall 307 is configured to allow a body of the syringe (not shown) to extend therethrough. The syringe receiving recess 302 also extends to an opening 308 in a side wall 309 of the main body 301. The opening 308 in the side wall 309 is configured to allow a syringe (not shown) to be inserted into the syringe receiving recess 302.

The main body 301 of the syringe connector 300 comprises a first portion 311 formed by a thermoplastic material. The first portion 311 is formed by the top, bottom, and side walls 305, 307, 309. The main body 301 further comprises a second portion 312. The second portion is formed by an inner layer 313 located on the inner surface of the second wall 307. The inner layer 313 is formed by an elastomeric material. The elastomeric inner layer 313 may be deformable and may grip a syringe when a syringe is received in the syringe receiving recess.

The first and second portions 311, 312 may be formed in a two-step injection moulding process. That is, the first portion 311 and may be formed in a first injection moulding process. Subsequently, at least one of the mould tools (not shown) is changed and the second portion 312 is formed in a second injection moulding process. The removal of a mould tool after the first moulding step and addition of a new mould tool to perform the second injection moulding step requires a complex tooling apparatus and increases the manufacturing time of the syringe connector 300.

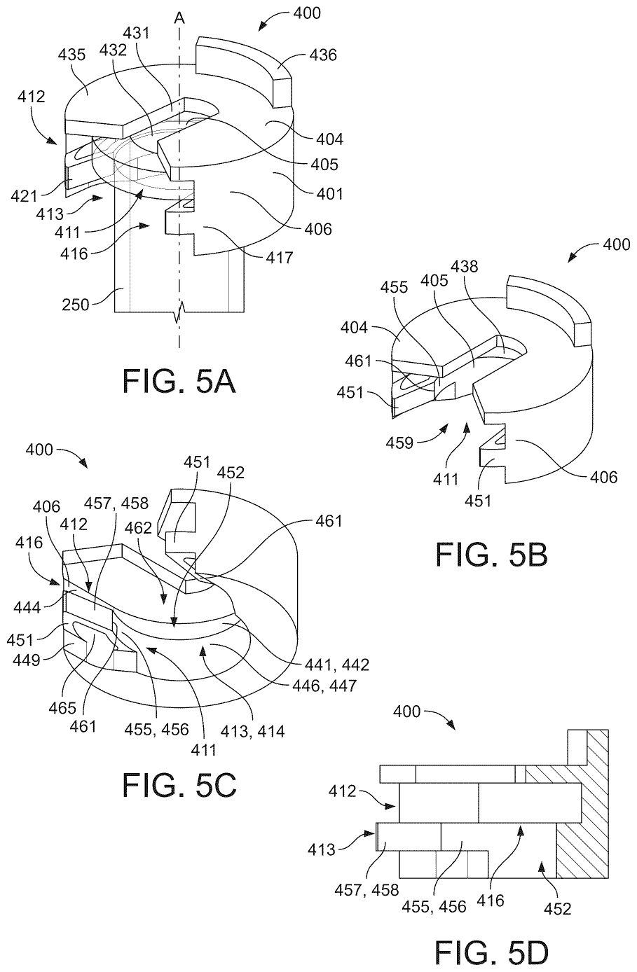

Referring now to FIGS. 5A to 13, schematic views of exemplary embodiments of a syringe connector 400 are shown. The syringe connector 400 comprises a main body 401. The main body 401 has a central longitudinal axis A. The main body 401 comprises a first wall 404. The first wall extends transversely to the longitudinal axis A. The main body 401 comprises a second wall 405. The second wall 405 opposes the first wall 404. The second wall 405 extends transversely to the longitudinal axis A. The main body 401 further comprises a side wall 406. The side wall 406 connects the first and second walls 404, 405. The side wall 406 may extends beyond the second wall 405.

The first wall 404, second wall 405, and the side wall 406 define a cavity 411 within the main body 401. The cavity 411 is configured to receive a proximal end of a syringe 250. The cavity 411 comprises a syringe flange receiving portion 412. The syringe flange receiving portion 412 is located between the first and second walls 404, 405 and delimited by the side wall 406. The cavity 411 further comprises a syringe body receiving position 413. The syringe body receiving portion 413 is formed by an aperture 414 extending through the second wall 405. The cavity 411 extends to an opening 416. The opening 416 is located in the side wall 406 and an edge 417 of the second wall 405. The opening 416 is configured such that a proximal end of a syringe 250 can be inserted transversely into the cavity 411.

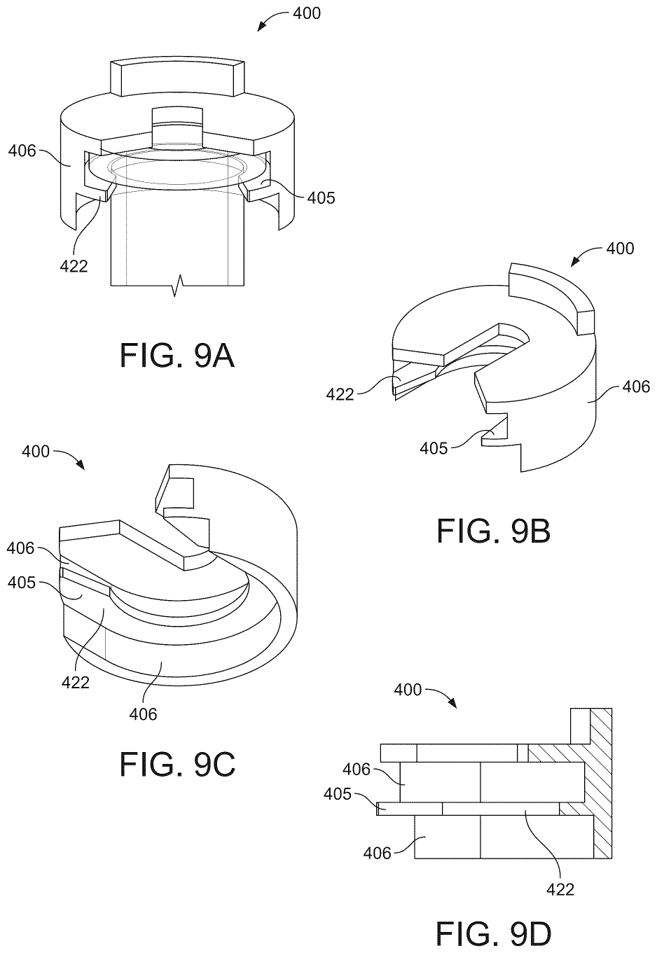

The syringe connector 400 further comprises a syringe retention element 421, shown in FIGS. 5A to 8D The syringe retention element 421 is configured to restrict radial movement of a syringe 250 when a syringe is located in the syringe connector 400. The syringe connector 400 further comprises an impact force reduction element 422, shown in FIGS. 9A to 11D. The impact force reduction element 422 is configured to reduce force transmitted to a syringe 250 when a dispensing mechanism 229 is triggered, during use of a medicament delivery device 200.

In the illustrated embodiments, the syringe retention element 421 and an impact force reduction element 422 are depicted separately for the purposes of clarity only. It will be appreciated that the syringe connector 400 may comprise any one of the syringe retention elements 421 disclosed herein, as shown in FIGS. 5A to 8D, in combination with any one of the impact force reduction elements 422 discloses herein, as shown in FIGS. 9A to 11D.

In some embodiments, the components of the syringe connector 400 may be integrally formed. That is, the components of the syringe connector 400 may be formed as a monolithic structure. The components of the syringe connector 400 may be an integrally formed monolithic structure. That is, all of the components may be formed from a single material without any fixing means, adhesives, or welds being required to hold the components together. The syringe connector 400 may be formed by an injection moulding process that comprises a single material injection step.

Advantageously, the components of the syringe connector 400 being formed integrally lowers the unit cost. In addition, the manufacture process is quicker due to only requiring one injection moulding step and removes the need for sub-assembly apparatus.

In some embodiments, the syringe connector 400 may be formed from a thermoplastic material. The thermoplastic material may comprise the material strength required to maintain the structure of the syringe connector 400 whilst providing the flexibility required to allow the syringe retention element to resiliently deform and the impact force reduction element to deform to absorb impact energy, and be useable in an injection moulded process. In some embodiments, the syringe connector 400 may be formed from an elastomeric material. The elastomeric material provides the same advantages as the thermoplastic material with enhanced impact energy absorbing properties.

Referring now to FIGS. 5A to 5D, a first embodiment of syringe retention element 421 of the syringe connector 400 is shown. The main body 401 of the syringe connector 400 may be generally cylindrical. Thus, the side wall 406 may extend parallel to the longitudinal axis A of the main body 401. In the present embodiment, the side wall 406 may be a generally circumferential wall.

The first wall 404 may comprise a central aperture 431 extending therethrough in the longitudinal direction. The central aperture 431 may be configured to allow a plunger rod (not shown) to extend therethrough to contact a piston (not shown) in a syringe 250 when a syringe is received in the syringe connector 400. The first wall 404 may further comprise a radially extending slot 432. The radially extending slot 432 may extend from the central aperture 431 to the outer edge of the first wall 404.

The first wall 404 may comprise a first contact surface 435. The first contact surface 435 may be an external surface of the first wall 404. The first contact surface 435 may be configured contact a spring guide (not shown) of a medicament delivery device 200, during use. The first contact surface 435 may comprise a longitudinally extending projection 436. The projection 436 may be configured to engage a recess (not shown) in the spring guide (not shown) in order to rotationally lock the spring guide to the syringe connector 400. The projection 436 may extend from the periphery of the first contact surface 435. Therefore, the projection 436 may extend circumferentially about the periphery of the first wall 404. The projection 436 may be located on the opposing side of the first wall 404 to the slot 432.

The side wall 406 may extend from a periphery of the first wall 404 to a periphery of the second wall 405. Thus, the second wall 405 may comprise an internal transverse surface 438. The internal transverse surface 438 of the second wall 405 may be configured to abut a flange 251 of a syringe 250, shown in FIG. 5A, received in the cavity 411 of the main body 401 of the syringe connector 400, when a syringe 250 is received therein. Thus, the internal transverse surface 405 may be configured to support a syringe 250 when received in the cavity 411.

The side wall 406 may comprise an internal circumferential surface portion 441, best shown in FIG. 5C. The internal circumferential surface portion 441 of the side wall 406 may form a closed end 442 of the cavity 411 in the transverse direction. The internal circumferential surface portion 441 of the side wall 406 may form a semi-circular closed end 442. That is, the internal circumferential surface portion 441 of the side wall 406 may be semi-circular or extend 180 degrees.

The side wall 406 may further comprise linear internal surface portions 444. The internal linear surface portions 444 may extend from opposing ends of the internal circumferential surface portion 441 of the side wall 406. The internal linear surface portions 444 may extend from the internal circumferential surface portion 441 to the opening 416 in the side wall 406. The internal linear surface portions 444 may extend parallel to each other. The internal linear surface portions 444 may extend parallel to the radial direction extending equidistantly therebetween. Thus, the internal circumferential surface portion 441 and the internal linear surface portions 444 of the side wall 406 between the first and second walls 404, 405 may define the syringe flange receiving portion 412 of the cavity 411.

The aperture 414 forming the syringe body receiving portion 413 may be defined by an internal circumferential surface portion 446. The internal circumferential surface portion 446 of the second wall 405 may form a closed end 447 in the transverse direction. The internal circumferential surface portion 446 of the second wall 405 may form a semi-circular closed end 447. That is, the internal circumferential surface portion 446 of the second wall 405 may be semi-circular or extend 180 degrees.

The aperture 414 forming the syringe body receiving portion 413 of the cavity 411 may be further defined by internal second surface portions 449. The internal second surface portions 449 may extend from opposing ends of the internal circumferential surface portion 446. The internal second surface portions 449 may extend from the internal circumferential surface portion 446 to the opening 416 at the edge of the second wall 405. The internal second surface portions 449 may extend generally radially. The internal second surface portions 449 may be arcuate. The internal second surface portions 449 may form a concave surface.

The outer end of the concave internal second surface portions 449 may align longitudinally with the outer end of the internal linear surface portions 444 of the side wall 406 at the opening 416.

The syringe retention element 421 may comprise a pair of arms 451. The pair of arms 451 may form a syringe clip. The arms 451 may be located on opposing sides of the cavity 411. The arms 451 may extend from the main body 401 into the cavity 411. The pair of arms 451 may define a syringe retention area 452 within the cavity 411. The syringe retention area 452 may be located centrally within the main body 401 of the syringe connector 400.

Advantageously, the syringe retention area 452 formed by the syringe clip arms 451 prevents a syringe 250 located within the syringe connector 400 from being accidentally removed and retains the syringe 250 within the syringe connector during use, as shown in FIG. 5A, especially during impact of the spring guide on the syringe connector 400 when the dispensing mechanism 229 is triggered.

The pair of arms 451 may be configured such that a minimum distance between the arms 451 is smaller than the diameter of the syringe retention area 452. Advantageously, this prevents a syringe 250 received in the syringe retention area 452 of the cavity 411 from being removed unless a predetermined force threshold is overcome to deform the arms 451 to separate such that the minimum distance between the arms 451 is at least as wide as the diameter of the syringe 250.

Each arm 451 of the pair of arms may be resiliently flexible. Each arm 451 may be resiliently flexible in an outward direction such that the arms 451 deform when a syringe is moved through the opening 416 into the syringe retention area 452. The pair of arms 451 may extend from an inner surface of the second wall 405 into the cavity 411. The pair of arms 451 are configured to contact a syringe body 252 to retain a syringe 250 when a syringe is received in the syringe retention area 452.

Each arm 451 may be in an initial position, as shown in FIGS. 5A to 5D, when before a syringe 250 is inserted into the syringe retention area 452 of the cavity 411. Each arm 451 may be deformed into a second position, wherein at least a portion of each arm 451 is moved further from the central longitudinal axis A of the main body 401, as the syringe 250 is moved through the opening 116 and into the syringe retention area 452. When the syringe 250 is received in the syringe retention area 452, each arm 451 may be biased back towards the initial position. The biasing force may be provided by the material properties of the arms 451. In some embodiments, when the syringe 250 is received in the syringe retention area 452, each arm 451 may move back to its initial position. In some embodiments, when the syringe 250 is received in the syringe retention area 452, each arm 451 may be prevented from moving back to its initial position by the syringe 250. However, the internal biasing force of each arm 451 may cause the arms 451 to return to an intermediate position between the initial position and the second position.

Each arm of the pair of arms 451 may comprise a radially innermost section 455. The radially innermost section 455 of each arm 451 may be configured to abut a side of a syringe 250 located proximal to the opening 416, when a syringe 250 is received in the syringe retention area 452. The radially innermost section 455 of the pair of arms 451 may be configured to restrict radial movement of a syringe 250, when a syringe is received in the syringe retentional area 452. The radially innermost section 455 of each arm 451 may comprise an inner surface 456. The inner surface 456 may be concave. The concave inner surface 456 of the radially innermost section 455 of each arm 451 may be contoured such that it is the same shape as the syringe body against which it abuts when a syringe 250 is received in the syringe retention area 452.

Advantageously, the inner surface 456 of the arm 451 being contoured to match the syringe body reduces the amount of rattle of the syringe 250 in the syringe connector 400. In addition, the contact area between the syringe 250 and the syringe connector 400 is increased, which minimises contact pressure on the syringe and reduces the likelihood of the syringe 250 becoming damaged.

Each arm of the pair of arms 451 may comprise a second section 457. The second section 457 may be located radially outwards of the innermost section 455 of each arm 451. The second section 457 of each arm 451 may extend away from the syringe retention area 452. Each second section 457 may comprise a guide surface 458. The guide surfaces 458 of each arm 451 may generally face toward each other and form a guide channel 459 therebetween. The guide surfaces 458 may be planar surfaces. The guide channel 459 may extend from the syringe retention area 452 to the opening 416 in the main body 401 of the syringe connector 400. The guide surfaces 458 may be configured to guide a syringe 250 along the guide channel 459 towards the syringe retention area 452 during syringe insertion. The outermost section of the second section 458 of each arm 451 may extend out of the opening 416 in the main body 401 of the syringe connector 400.

Advantageously, the guide surfaces 458 may result is a larger positional tolerance for assembly of the syringe in the syringe connector. That is, the guide surfaces remove the low tolerance on accuracy required for assembly.

The innermost section 455 and second section 457 of each arm 451 join at a ridge 461. That is, each arm 451 comprises a ridge 461 where the innermost section 455 of the arm 451 and the second section 457 of the arm 451 meet. The ridge 461 may be formed by a pointed edge, a rounded edge, or a pointed or rounded bump or projection. A gap 462 between the opposing ridges 461 may be the minimum distance between the arms 451 of the syringe clip. The guide surfaces 458 of the second sections 457 of the arms 451 may diverge with distance from the ridges 461 in the radial direction. That is, the gap between the outermost part of the second sections 458 is larger than the gap between the ridges 461.

Advantageously, the ridge 461 may help to retain the syringe 250 in the syringe retention area 452 to prevent, or at least reduce the likelihood, of accidentally decoupling of the syringe 250 and the syringe connector 400.

In the present embodiment, the innermost section 455 and the second section 458 of each arm 451 both extend from the inner surface of the second wall 405. That is, the innermost section 455 and the second section 458 of each arm 451 both extend from the internal second surface portions 449 of the second wall 405. Thus, the innermost section 455 and the second section 458 of each arm 451 may form an enclosed aperture 465 between each arm 451 and the second wall 405. Each arm 451 may be configured to deform into its respective aperture 465 when a syringe 250 is inserted into the syringe retention area 452.

Advantageously, the enclosed aperture 465 may reduce the force required to deflect the arms 451 during coupling of the syringe 250 to the syringe connector 400. By reducing this assembly force, the chance of causing damage to the syringe or permanent deformation of the syringe connector can be reduced.

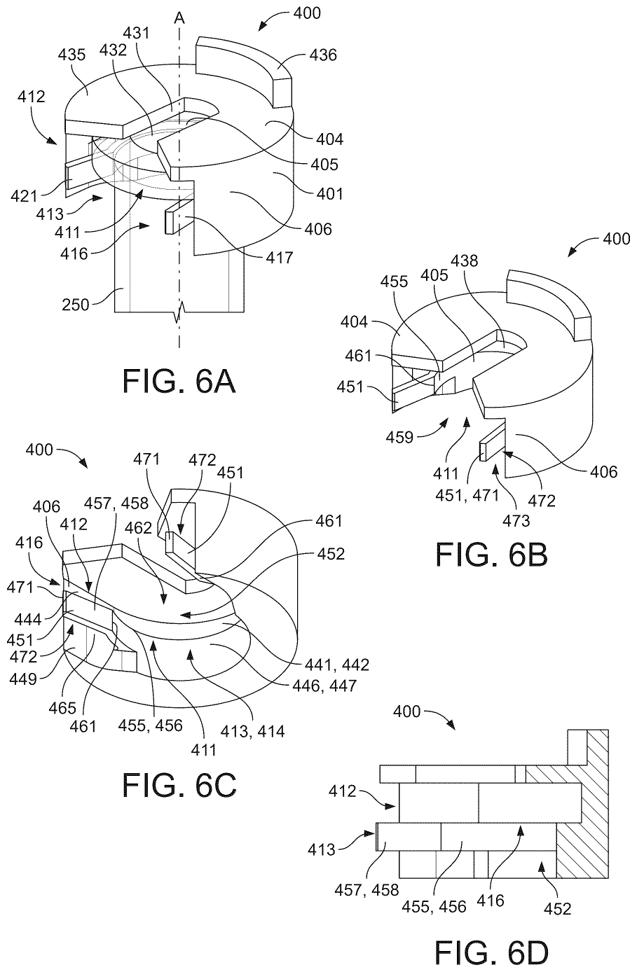

Referring now to FIGS. 6A to 6D, another embodiment of the syringe retention element 421 of the syringe connector 400 is shown. The syringe retention element 421 illustrated in FIGS. 6A to 6D is generally the same as the syringe retention element 421 previously described with reference to FIGS. 5A to 5D. Therefore, a detailed description of the syringe retention element 421 and syringe connector 400 will be omitted herein for brevity. Furthermore, similar features and components of the embodiment of the syringe retention element 421 and syringe connector 400 will retain the same terminology and reference numerals. The main difference between the syringe retention element 421 of the present embodiment and the syringe retention element 421 of FIGS. 5A to 5D is the configuration of the pair of arms 451.

In the present embodiment, the innermost section 455 of each of the pair of arms 451 may extend from the inner surface of the second wall 405. That is, the innermost section 455 of each arm 451 extends from the internal second surface portions 449 of the second wall 405. The second section 458 of each arm 451 may comprise a free-end 471. An open ended slot 472 may be located between each arm 451 and the second wall 405. An open end 473 of each slot 472 is located distal to and facing away from the syringe retention area 452. Each arm 451 may be configured to deform into its respective slot 472 when a syringe 250 is inserted into the syringe retention area 452.

Advantageously, the open-ended slot 472 may provide an even greater reduction in deflection force. Thus, the ease of assembly is increased and the chance of damaging the syringe or permanently deforming the arm 451 is reduced. The tooling for such an embodiment is also easier to produce and can be moved in the axial direction or the radial direction during the manufacturing process due to the open-ended slot 472.

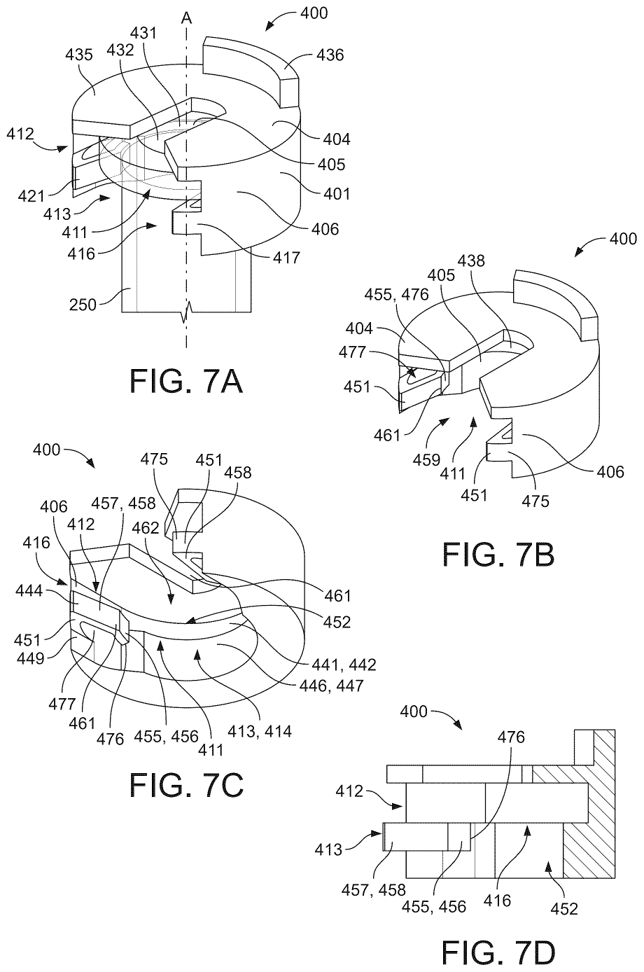

Referring now to FIGS. 7A to 7D, another embodiment of the syringe retention element 421 of the syringe connector 400 is shown. The syringe retention element 421 illustrated in FIGS. 7A to 7D is generally the same as the syringe retention element 421 previously described with reference to FIGS. 5A to 6D. Therefore, a detailed description of the syringe retention element 421 and syringe connector 400 will be omitted herein for brevity. Furthermore, similar features and components of the embodiment of the syringe retention element 421 and syringe connector 400 will retain the same terminology and reference numerals. The main difference between the syringe retention element 421 of the present embodiment and the syringe retention element 421 of FIGS. 5A to 6D is the configuration of the pair of arms 451.

In the present embodiment, the second section 458 may extend from the inner surface of the second wall 405. The second section 458 may comprise a circumferential portion that extends from the second wall 405. That is, the circumferential portion 475 of the second section 458 of each arm 451 extends from the internal second surface portions 449 of the second wall 405. The innermost section 455 may comprise a free end 476. An open ended slot 477 may be located between each arm 451 and the second wall 405. An open end 478 of the slot 477 may be proximate to and facing toward the syringe retention area 452. Each arm 451 may be configured to deform into its respective slot 477 when a syringe is inserted into the syringe retention area 452.

Advantageously, the open-ended slot 477 may provide a greater reduction in deflection force. Thus, the ease of assembly is increased and the chance of damaging the syringe or permanently deforming the arm 451 is reduced.

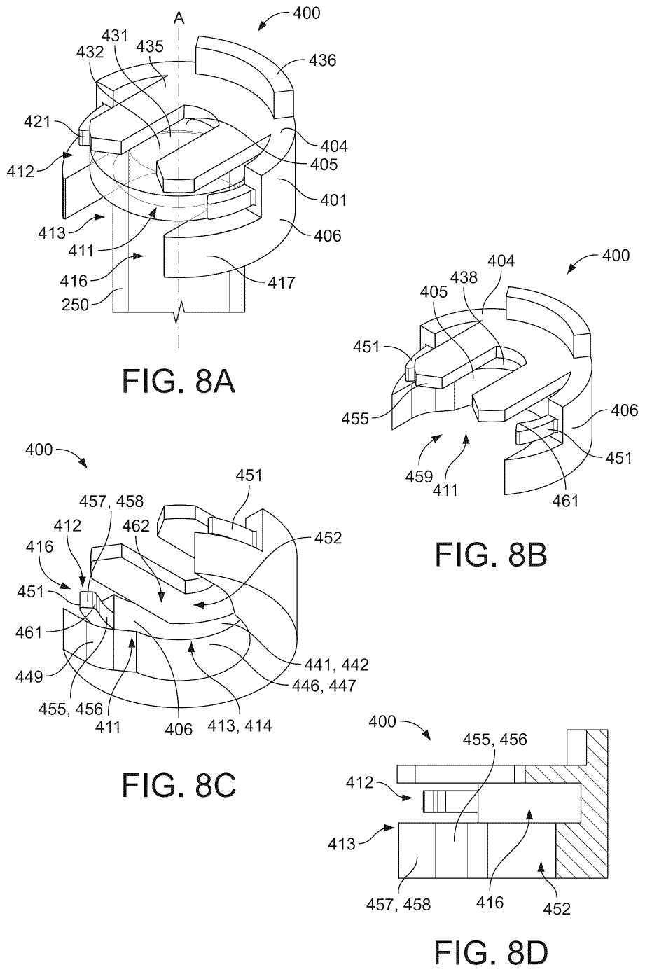

Referring now to FIGS. 8A to 8D, another embodiment of the syringe retention element 421 of the syringe connector 400 is shown. The syringe retention element 421 illustrated in FIGS. 8A to 8D is generally the same as the syringe retention element 421 previously described with reference to FIGS. 5A to 7D. Therefore, a detailed description of the syringe retention element 421 and syringe connector 400 will be omitted herein for brevity. Furthermore, similar features and components of the embodiment of the syringe retention element 421 and syringe connector 400 will retain the same terminology and reference numerals. The main difference between the syringe retention element 421 of the present embodiment and the syringe retention element 421 of FIGS. 5A to 7D is the configuration of the pair of arms 451.

In the present embodiment, the pair of arms 451 may extend from the side wall 406 into the syringe flange receiving portion 412 of the cavity 411. The pair of arms 451 may be configured to contact a syringe flange 251. The pair of arms 451 may be configured to retain a syringe 250 when a syringe 250 is received in the syringe retention area 452.

In the present embodiment, the innermost section 455 may be formed by the inner surface 456 of each arm 451. The second section 457 may be formed by a guide surface 458 adjacent to the inner surface 456 of each arm 451.

Referring now to FIGS. 9A to 11D, embodiments of the impact force reduction element 422 are shown. As previously disclosed, any of the embodiments of the impact force reduction element 422 may be used with any of the syringe retention elements 421 previously described.

Referring now to FIGS. 9A to 9D, the impact force reduction element 422 may be formed by the second wall 405. In such an embodiment, the second wall 405 may be resiliently deformable in the longitudinal direction. That is, the second wall 405 may deflect about the point where it joins the side wall 406. The second wall 405 may be resiliently deformable in the longitudinal direction to absorb impact energy during use. That is, as the second wall 405 is deformable, energy is required to deform the second wall 405.

As a result, when the dispensing mechanism 229 is triggered, the biasing member forces the spring guide into the syringe connector 400 and subsequently the syringe connector 400 into a collar, the second wall 405 can be deformed to absorb some of the energy released by the extending spring when the second wall 405 of the syringe connector 400 is driven into the collar.

In some embodiments, the second wall 405 may be dimensioned such that it readily deformable. That is, the second wall 405 may have a thickness in the range of between about 0.5 mm and about 2.0 mm.

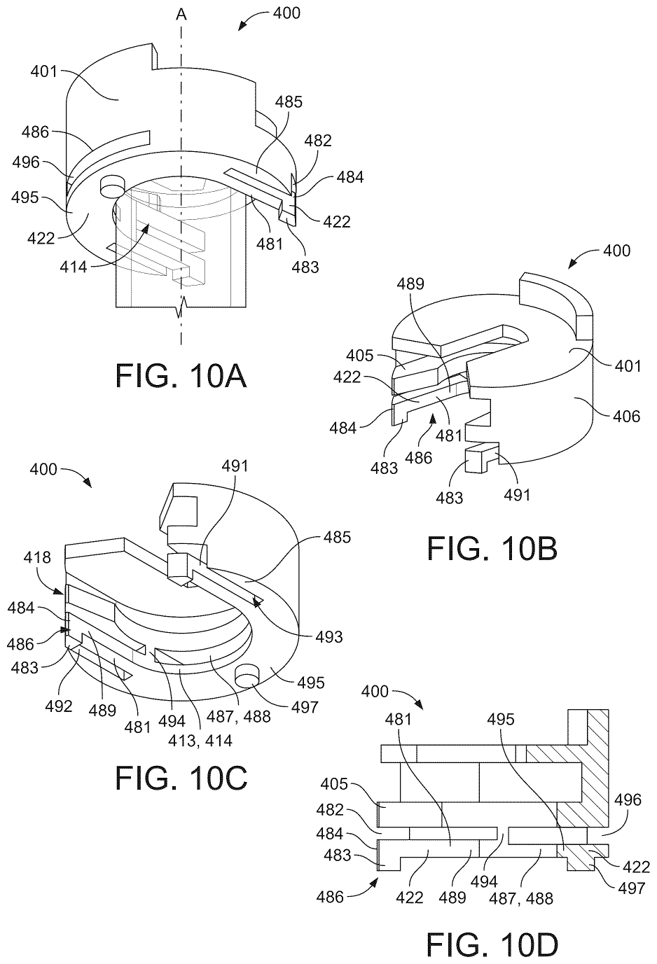

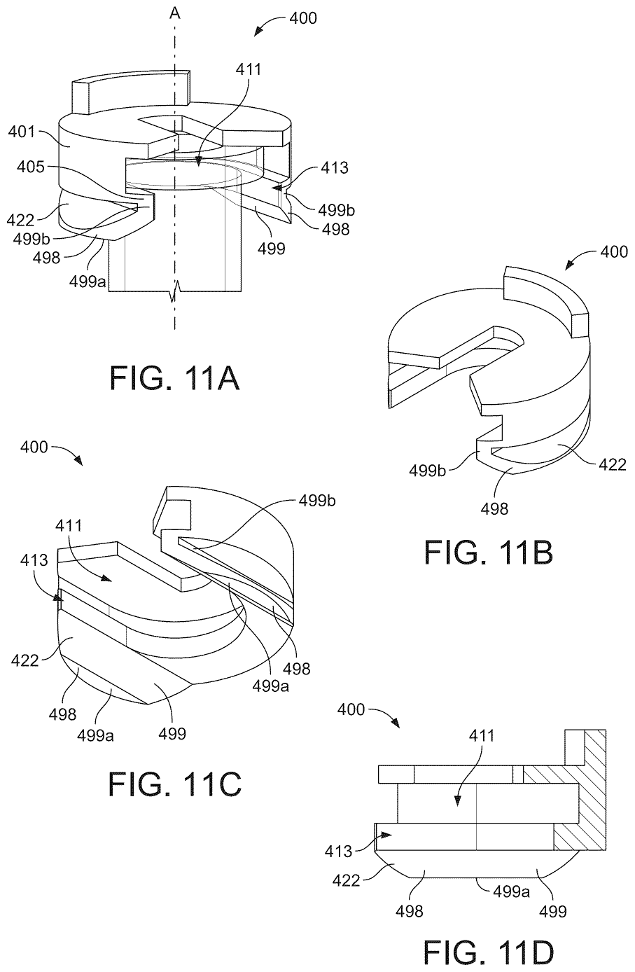

Referring now to FIGS. 10A to 11D, the impact force reduction element 422 may be located outside of the main body 401 of the syringe connector 400. That is, the impact force reduction element 422 may be located outside of the main body 401 longitudinally adjacent to and extending from the second wall 405. The impact force reduction element 422 may be configured to resiliently deform to absorb impact energy, during use.

Referring to FIGS. 10A to 10D, the impact force reduction element 422 may comprise at least one transversely extending cantilevered arm 481. The at least one transversely extending cantilevered arm 481 may be longitudinally spaced from the second wall 405. The at least one transversely extending cantilevered arm 481 may be longitudinally spaced from the second wall 405 by a transversely extending slot 482.

The at least one cantilevered arm 481 may comprise a contact projection 483. The contact projection 483 may be located distally to the longitudinal axis A of the main body 401. That is, the contact projection 483 may be located on a free-end 484 of the cantilevered arm 481. The contact projection 483 may extend in the longitudinal direction away from the main body 401 of the syringe connector 400. The contact projection 483 may be configured to abut a component of a medicament delivery device 200, such as a collar, during use. The projection 483 may be configured to ensure that the component only contacts the projection 483, and does not contact the main body of cantilevered arm 481, during bending of the cantilevered arm 481. Thus, the free-end of the cantilevered arm 481, where the projection 483 is located, is free to bend through a larger range without the force being directly transferred to main body of the cantilevered arm 481. This

In the present embodiment, the side wall 406 may extend longitudinally beyond the second wall 405. It will be appreciated that the second wall 405 is illustrated simply without any detail relating to the syringe retention element 421. A transversely extending third wall 485 may extend from a distal end 486 of the side wall 406. The third wall 485 may have generally the same footprint, i.e. cross-sectional outline in a plane perpendicular to the longitudinal axis A, as the second wall 405 with a few minor differences.

That is, the aperture 414 that extends through the second wall 405 may also extend through the third wall 485 such that the syringe body receiving portion 413 of the cavity 411 of the main body is further defined by the third wall 485. The aperture 414 in the third wall 485 may also extend radially to an opening 486. The opening 486 in the third wall 485 may be circumferentially aligned, i.e. longitudinally adjacent to, the opening 416 in the second and side walls 405, 406.

The aperture 414 in the third wall 485 may be defined by an internal circumferential surface portion 487. The internal circumferential surface portion 487 of the third wall 485 may form a closed end 488 in the transverse direction. The internal circumferential surface portion 487 of the third wall 485 may form a semi-circular closed end 488. That is, the internal circumferential surface portion 487 of the third wall 485 may be semi-circular or extend 180 degrees.

The aperture 414 of the syringe body receiving portion 413 in the third wall 485 may be further defined by internal second surface portions 489. The internal second surface portions 489 may extend from opposing ends of the internal circumferential surface portion 487. The internal second surface portions 489 may extend from the internal circumferential surface portion 487 to the opening 416 at the edge of the third wall 485. The internal second surface portions 489 may extend linearly from the internal circumferential surface portion 487. That is, the internal second surface portion 499 may extend tangentially to the circumferential surface portion 487.

In the present embodiment, the internal second surface portions 489 may be formed by the inner surfaces of opposing cantilevered arms 481. An outer surface 491 of each cantilevered arm 481 may be spaced from an inner longitudinal surface 492 of the third wall 485 by a groove 493. The internal second surface portion 489 and the inner longitudinal surface 492 may extend parallel to each other. Thus, each cantilevered arm 481 may be linear in the transverse direction.

The cantilevered arm 481 may be supported by an anchor 494. The anchor 494 may be located at the opposite end of the cantilevered arm 481 to the projection 483 or free-end 484 of the cantilevered arm 481. The anchor 494 may connect the cantilevered arm 481 to the second and third walls 405, 485. The anchor 494 may at least partially define the aperture 414 of the cavity 411 and may define an end of the slot 482 between the second and third walls 405, 485. The anchor 494 may be located at roughly the same distance from the opening 418 as the central longitudinal axis A of the main body 401 of the syringe connector 400 that passes through the centre of the syringe retention area 452.

In the present embodiment, the internal circumferential surface portion 487 of the third wall 485 may also be formed by the inner surface of a circumferentially extending cantilevered arm 495. The circumferentially extending cantilevered arm 495 may extend from the anchor 494 on one side of the cavity 411 to the anchor 494 located on the other side of the cavity 411. The circumferentially extending cantilevered arm 495 may be a portion of the third wall 485 that is separated from the second wall 405 may a second transversely extending slot 496. The circumferentially extending cantilevered arm 495 may comprise a contact projection 497 that is located mid-way between the anchors 494 in the circumferential direction.

The use of three cantilevered arms 481 located substantially equidistantly around the syringe retention area 452 improves impact absorption when the syringe connector is biased into the collar by the spring. As the force from the impact is evenly distributed around the syringe connector 400, the potential alignment issues of the syringe during use are alleviated.

Referring to FIGS. 11A to 11D, the impact force reduction element 422 may comprise a pair of deformable arms 498. The pair of deformable arms 498 may extend from opposing sides of the syringe body receiving portion 413 of the cavity 411. The arms 498 may extend parallel to each other. Each arm 498 of the pair of arms 498 may comprise an inclined portion 499. The distance of the inclined portion 499 the from the longitudinal axis of the main body 401 may increase with distance from the second wall 405. A free-end 499a of each arm 498 may comprise a transverse surface configured to abut a component of a medicament delivery device 200, such as a collar, during use. Each arm 498 may be connected to the second wall 405 by a support 499b about which the arm 498 may rotate and/or bend when under load to absorb impact energy.

Referring back to FIG. 4, a schematic cross-sectional side view of an exemplary moulding apparatus 500 for forming a syringe connector 400 is shown. The moulding apparatus 500 comprises first, second, and third mould parts 501, 502, 503.

The first wall 404 of the syringe connector 400 may be defined by a first gap 505 between the first and second mould parts 501, 502. The second wall 405 of the syringe connector 400 may be defined by a second gap 506 between the second and third mould parts 502, 503. The syringe retention element 421 and the impact force reduction element 422 may also be defined by the shape of the third mould part 503. The syringe flange receiving portion 412 of the cavity 411 may be defined by the second mould part 502. The syringe body receiving portion 413 of the cavity 411 may be defined by the third mould part 503. The side wall 406 may be defined by a third gap 507 between the first, second, and third mould parts 501, 502, 503.

To form a syringe connector 400, the mould parts 501, 502, 503 may be positioned together to form a syringe connector shaped cavity 508. A material injection port 509 may be provided in at least one of the mould parts 501, 502, 503. A material may then be injected into the syringe connector shaped cavity 508 through the injection port 509. This step may be performed as a single injection step to form all of the components and/or features of the syringe connector 400 in one injection step. The material may then be cooled to form the syringe connector 400. Once cooled, the newly formed syringe connector 400 may be removed after the mould parts 501, 502, 503 have been separated.

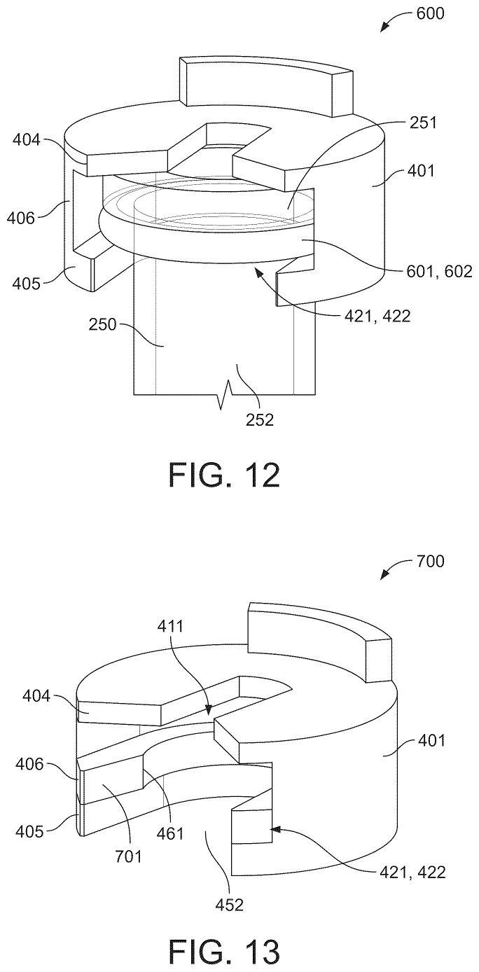

Referring now to FIG. 12, another embodiment of the syringe connector 600 is shown. The syringe connector 600 is generally the same as the syringe connector 400 previously described with reference to FIGS. 5A to 11D. Therefore, a detailed description of the syringe connector 600 will be omitted herein for brevity. Furthermore, similar features and components of the embodiment of the syringe connector 600 will retain the same terminology and reference numerals.

The main difference between the syringe connector 600 of the present embodiment and the syringe connector 400 of FIGS. 5A to 11D is that the syringe connector 600 is not a monolithic structure. On the contrary, the syringe connector 600 comprises a main body 401 formed by a first wall 404, a second wall 405, and a side wall 406, as previously described. However, an insert 601 is provided that is located in the cavity 411 of the syringe connector 600. The insert 601 forms the syringe retention element 421 and the impact force reduction element 422.

In FIG. 12, the insert 601 is in the form of an O-ring 602. The O-ring 602 is configured to abut the inner transverse surface of the second wall 405 and the inner circumferential surface of the side wall 406, as well as the body 252 of the syringe 250. The O-ring may abut the flange 251 of the syringe 250. The interference fit of the O-ring 602 within the cavity 411 and to the body 252 of the syringe 250 forms the syringe retention element 421 to prevent radial movement of the syringe 250 in the main body 401 of the syringe connector 600. The O-ring 602 may be formed of an elastomeric material that is configured to deform under load. Thus, the O-ring being formed by an elastomeric material provides the O-ring with the impact force reduction element 422.

Referring now to FIG. 13, another embodiment of the syringe connector 700 is shown. The syringe connector 700 is generally the same as the syringe connector 600 previously described with reference to FIGS. 5A to 11D. Therefore, a detailed description of the syringe connector 700 will be omitted herein for brevity. Furthermore, similar features and components of the embodiment of the syringe connector 700 will retain the same terminology and reference numerals.

The main difference between the syringe connector 700 of the present embodiment and the syringe connector 400 of FIGS. 5A to 11D is that the syringe connector 700 is not a monolithic structure. On the contrary, the syringe connector 700 comprises a main body 401 formed by a first wall 404, a second wall 405, and a side wall 406, as previously described. However, an insert 701 is provided that is located in the cavity 411 of the syringe connector 700. The insert 701 forms the syringe retention element 421 and the impact force reduction element 422.

The insert 701 may be generally horseshoe shaped and configured to cover the inner transverse surface of the second wall 405. The insert 701 may comprise a pair of ridges 461 that extend into the cavity 411. The pair of ridges 461 may form the syringe retention element 421 and at least partially define a syringe retention area 452 in the cavity 411.

The insert 701 may be formed from an elastomeric material. Thus, the insert 701 may be deformable under load, and consequently formed as an impact force reduction element 422. The insert 701 may be formed in a separate single injection step moulding process and the inserted and connected to the main body 401 of the syringe connector 700. The insert 701 may be adhered to the second wall 405 of the syringe connector 700.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short-or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom. Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin. Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV). The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, small modular immunopharmaceuticals (SMIP), binding-domain immunoglobulin fusion proteins, camelized antibodies, and immunoglobulin single variable domains. Additional examples of antigen-binding antibody fragments are known in the art.

The term “immunoglobulin single variable domain” (ISV), interchangeably used with “single variable domain”, defines immunoglobulin molecules wherein the antigen binding site is present on, and formed by, a single immunoglobulin domain. As such, immunoglobulin single variable domains are capable of specifically binding to an epitope of the antigen without pairing with an additional immunoglobulin variable domain. The binding site of an immunoglobulin single variable domain is formed by a single heavy chain variable domain (VH domain or VHH domain) or a single light chain variable domain (VL domain). Hence, the antigen binding site of an immunoglobulin single variable domain is formed by no more than three CDRs.

An immunoglobulin single variable domain (ISV) can be a heavy chain ISV, such as a VH (derived from a conventional four-chain antibody), or VHH (derived from a heavy-chain antibody), including a camelized VH or humanized VHH. For example, the immunoglobulin single variable domain may be a (single) domain antibody, a “dAb” or dAb or a Nanobody® ISV (such as a VHH, including a humanized VHH or camelized VH) or a suitable fragment thereof. [Note: Nanobody® is a registered trademark of Ablynx N.V.]; other single variable domains, or any suitable fragment of any one thereof.

“VHH domains”, also known as VHHs, VHH antibody fragments, and VHH antibodies, have originally been described as the antigen binding immunoglobulin variable domain of “heavy chain antibodies” (i.e., of “antibodies devoid of light chains”; Hamers-Casterman et al. 1993 (Nature 363: 446-448). The term “VHH domain” has been chosen in order to distinguish these variable domains from the heavy chain variable domains that are present in conventional 4-chain antibodies (which are referred to herein as “VH domains”) and from the light chain variable domains that are present in conventional 4-chain antibodies (which are referred to herein as “VL domains”). For a further description of VHH's, reference is made to the review article by Muyldermans 2001 (Reviews in Molecular Biotechnology 74: 277-302).

For the term “dAb's” and “domain antibody”, reference is for example made to Ward et al. 1989 (Nature 341: 544), to Holt et al. 2003 (Trends Biotechnol. 21: 484); as well as to WO 2004/068820, WO 2006/030220, WO 2006/003388. It should also be noted that, although less preferred in the context of the present invention because they are not of mammalian origin, single variable domains can be derived from certain species of shark (for example, the so-called “IgNAR domains”, see for example WO 2005/18629).

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

An example of a compound to be administered with the drug delivery device disclosed herein is a compound with the INN tirzepatide, as referenced in claim 1 of U.S. Pat. No. 9,474,780.

An example of a pharmaceutical composition to be administered with the drug delivery device disclosed herein is a pharmaceutical composition as referenced in U.S. Pat. No. 11,357,820.

An example of a pharmaceutical composition to be administered with the drug delivery device disclosed herein includes a 0.5 mL solution of 2.5 mg, 5 mg, 7.5 mg, 10 mg, 12.5 mg, or 15 mg of tirzepatide and the following excipients sodium chloride (4.1 mg), sodium phosphate dibasic heptahydrate (0.7 mg), and water for injection. Hydrochloric acid solution and/or sodium hydroxide solution may be added to adjust the pH.

An example starting dosage tirzepatide may be 2.5 mg injected subcutaneously once weekly. After four weeks, the tirzepatide dosage may be increased to 5 mg injected subcutaneously once weekly. The dosage may be further increased in 2.5 mg increments after at least four weeks on the current dose. In an example, the maximum dosage of tirzepatide may be 15 mg injected subcutaneously once weekly. If a dose is missed, patients may be instructed to administer tirzepatide as soon as possible within four days (96 hours) after the missed dose. If more than four days have passed, patients may skip the missed dose and administer the next dose on the regularly scheduled day. In each case, patients may then resume their regular once weekly dosing schedule. The day of weekly administration may be changed, if necessary. The time between two doses may be at least three days (72 hours).

Tirzepatide dosages may include 2.5 mg/0.5 mL, 5 mg/0.5 mL, 7.5 mg/0.5 mL, 10 mg/0.5 mL, 12.5 mg/0.5 mL, and 15 mg/0.5 mL. Tirzepatide may be stored in a refrigerator at 2° C. to 8° C. (36° F. to 46° F.). A single-dose pen or single-dose vial may be stored unrefrigerated at temperatures not to exceed 30° C. (86° F.) for up to 21 days. Tirzepatide may be stored in a carton.

### LIST OF REFERENCE NUMERALS

10—Device
11—Housing
12—Cap
13—Needle Sleeve
17—Needle
20—Distal Region
21—Proximal Region
22—Button
23—Piston
200—Medicament Delivery Device
201—Body
202—Distal End
208—Locking Member
216—Lock Ring
217—Needle
223—Plunger
227—Actuation Member
228—Button
229—Dispensing Mechanism
232—Injection Site
240—Spring Guide
242—Protrusions
250—Syringe
251—Flange
252—Body
260—Spring
262—Spring
264—Clip
265—Proximal Opening
266—Needle Shield
267—Collar
268—Collar
400—Syringe Connector
401—Main Body
404—First Wall
405—Second Wall
406—Side Wall
411—Cavity
412—Syringe Flange Receiving Portion
413—Syringe Body Receiving Portion
414—Aperture
416—Opening
417—Edge
421—Syringe Retention Element
422—Impact Force Reduction Element
431—Central Aperture
432—Slot
435—First Contact Surface
436—Projection
438—Internal Transverse Surface
441—Internal Circumferential Surface Portion
442—Semi-Circular Closed End
444—Internal Linear Surface Portion
446—Internal Circumferential Surface Portion
447—Semi-Circular Closed End
449—Internal Second Surface Portion
451—Pair of Arms
452—Syringe Retention Area
455—Radially Innermost Section
456—Inner Surface
459—Guide Channel
461—Ridge
462—Gap
465—Enclosed Aperture
471—Free End
472—Slot
473—Open End
475—Circumferential Portion
476—Free End
477—Slot
478—Open End
481—Cantilevered Arm
482—Slot
483—Contact Projection
484—Free End
485—Third Wall
486—Opening
487—Internal Circumferential Wall
488—Closed End
489—Internal Second Surface Portion
491—Outer Surface
492—Inner Longitudinal Surface
493—Groove
494—Anchor
495—Circumferentially Extending Cantilevered Arm
496—Slot
497—Contact Projection
498—Pair of Arms
499—Inclined Portion
499a—Free-End
499b—Support
500—Moulding Apparatus
501—First Mould Part
502—Second Mould Part
503—Third Mould Part
507—Third Gap
508—Syringe Connector Shaped Cavity
509—Injection Port
600—Syringe Connector
601—Insert
602—O-Ring
700—Syringe Connector
701—Insert

1-20. (canceled)

21. A syringe connector for a medicament delivery device, the syringe connector comprising:
a main body having a longitudinal axis, the main body comprising a first wall extending transversely to the longitudinal axis, an opposing second wall extending transversely to the longitudinal axis, and a side wall connecting the first wall and the second wall, the first wall and the second wall defining a cavity configured to receive a proximal end of a syringe,
wherein the cavity comprises a syringe flange receiving portion located between the first wall and the second wall and delimited by the side wall, and a syringe body receiving portion formed by an aperture extending through the second wall, and
wherein the cavity extends to an opening in the side wall and an edge of the second wall such that a proximal end of a syringe can be inserted transversely into the cavity;
a syringe retention element configured to restrict radial movement of the syringe when the syringe is located in the syringe connector, wherein the syringe retention element comprises a pair of arms that project radially inward from the side wall into the syringe flange receiving portion of the cavity, the pair of arms being positioned longitudinally between the first wall and the second wall and separate from both the first wall and the second wall, each arm of the pair of arms having a bottom surface positioned proximally relative to a top surface of the second wall along the longitudinal axis and spaced from the top surface of the second wall by a gap such that the arm is deflectable in a radial direction within the syringe flange receiving portion; and
an impact force reduction element configured to reduce force transmitted to the syringe when a dispensing mechanism is triggered during use.

22. The syringe connector according to claim 21, wherein the pair of arms form a syringe clip, the arms being located on opposing sides of the cavity and extending from the main body into the cavity to define a central syringe retention area within the cavity.

23. The syringe connector according to claim 22, wherein the pair of arms is configured such that a minimum distance between the arms is smaller than a diameter of the syringe retention area.

24. The syringe connector according to claim 22, wherein each arm of the pair of arms is resiliently flexible in an outward direction such that the arms deform when a syringe is moved through the opening into the syringe retention area.

25. (canceled)

26. The syringe connector according to claim 22, wherein the pair of arms extend from the side wall into the syringe flange receiving portion and are configured to contact a syringe flange and retain a syringe when a syringe is received in the syringe retention area.

27. The syringe connector according to claim 22, wherein each arm of the pair of arms comprises a radially innermost section configured to abut a side of the syringe proximal to the opening and restrict radial movement of the syringe when the syringe is received in the syringe retention area.

28. The syringe connector according to claim 27, wherein each arm of the pair of arms comprise a second section located radially outwards of the innermost section, each second section comprising a guide surface configured to guide a syringe towards the syringe retention area during syringe insertion.

29. The syringe connector according to claim 28, wherein the pair of arms is configured such that the innermost section and the second section of each arm join at a ridge, and a gap between opposing ridges being the minimum distance between the arms, wherein the guide surfaces are configured to diverge with distance in the radial direction.

30. The syringe connector according to claim 28, wherein the pair of arms is configured such that the innermost section and the second section of each arm both extend from the inner surface of the second wall to form an enclosed aperture between each arm and the second wall, each arm being configured to deform into its respective aperture when the syringe is inserted into the syringe retention area.

31. The syringe connector according to claim 28, wherein the innermost section of each arm is configured to extend from the inner surface of the side wall and the second section of each arm comprises a free end, an open ended slot located between each arm and the second wall, wherein the open end of the slot is distal to and facing away from the syringe retention area, each arm being configured to deform into its respective slot when the syringe is inserted into the syringe retention area.

32. The syringe connector according to claim 28, wherein the second section of each arm is configured to extend from the inner surface of the side wall and the innermost section comprises a free end, an open ended slot located between each arm and the second wall, wherein the open end of the slot is proximate to and facing toward the syringe retention area, wherein each arm is configured to deform into its respective slot when the syringe is inserted into the syringe retention area.

33. The syringe connector according to claim 21, wherein the impact force reduction element is located outside of the main body longitudinally adjacent to and extending from the second wall, the impact force reduction element being configured to resiliently deform to absorb impact energy during use.

34. The syringe connector according to claim 33, wherein the impact force reduction element comprises at least one transversely extending cantilevered arm longitudinally spaced from the second wall by a transversely extending slot, the at least one cantilevered arm comprising a contact projection located distally to the longitudinal axis and extending in the longitudinal direction away from the main body.

35. The syringe connector according to claim 33, wherein the impact force reduction element comprises a pair of deformable arms extending from opposing sides of the syringe body receiving portion of the cavity, wherein the arms extend parallel to each other, and each arm of the pair of arms comprises an inclined portion whose distance from the longitudinal axis of the main body increases with distance from the second wall.

36. The syringe connector according to claim 21, wherein the impact force reduction element is formed by the second wall, wherein the second wall is resiliently deformable in the longitudinal direction to absorb impact energy during use.

37. The syringe connector according to claim 21, wherein components of the syringe connector are integrally formed as a monolithic structure.

38. The syringe connector according to claim 21, wherein the syringe connector is formed from a thermoplastic material or an elastomeric material.

39. A medicament delivery device comprising a syringe connector that comprises:
a main body having a longitudinal axis, the main body comprising a first wall extending transversely to the longitudinal axis, an opposing second wall extending transversely to the longitudinal axis, and a side wall connecting the first wall and the second wall, the first wall and the second wall defining a cavity configured to receive a proximal end of a syringe,
wherein the cavity comprises a syringe flange receiving portion located between the first wall and the second wall and delimited by the side wall, and a syringe body receiving portion formed by an aperture extending through the second wall, and
wherein the cavity extends to an opening in the side wall and an edge of the second wall such that a proximal end of a syringe can be inserted transversely into the cavity;
a syringe retention element configured to restrict radial movement of the syringe when the syringe is located in the syringe connector, wherein the syringe retention element comprises a pair of arms that project radially inward from the side wall into the syringe flange receiving portion of the cavity, the pair of arms being positioned longitudinally between the first wall and the second wall and separate from both the first wall and the second wall, each arm of the pair of arms having a bottom surface positioned proximally relative to a top surface of the second wall along the longitudinal axis and spaced from the top surface of the second wall by a gap such that the arm is deflectable in a radial direction within the syringe flange receiving portion; and
an impact force reduction element configured to reduce force transmitted to the syringe when a dispensing mechanism is triggered during use.

40. A method of forming a syringe connector for a medicament delivery device, the method comprising:
positioning a plurality of mold parts to form a syringe connector shaped cavity;
providing a material injection port in at least one of the plurality of mold parts;
performing a single injection of material into the syringe connector shaped cavity to fill the syringe connector shaped cavity; and
cooling the material to form the syringe connector.
